(19) United States
(12) Patent Application Publication
(10) Pub. No.: US 2020/0276045 A1
(43) Pub. Date: Sep. 3, 2020

(54) TEMPORARY STOMA EVACUATION DEVICE

(71) Applicant: TECHNION RESEARCH AND DEVELOPMENT FOUNDATION LIMITED, HAIFA (IL)

(72) Inventor: MARIANA BENDAVIT, TEL AVIV (IL)

(21) Appl. No.: 16/759,758

(22) PCT Filed: Oct. 29, 2018
(86) PCT No.: PCT/IL2018/051153

§ 371 Date: Apr. 28, 2020

Related U.S. Application Data
(60) Provisional application No. 62/578,452, filed on Oct. 29, 2017.

(51) Int. Cl. A61F 5/445; A61F 5/44

(52) U.S. Cl. CPC A61F 5/445; A61F 2005/4402; A61F 5/4408; A61F 5/4405

(57) ABSTRACT

A device for fixing in a stoma for collection of excrement, comprising a catheter, a balloon at the distal end of the device, and manual rotatable pump at the proximal end of the device. The pump has a lid that is rotatable in two directions, and the lid is attached to a paddle that moves through an annular air chamber. Rotation of the lid in one direction causes air to flow down a tube and inflate the balloon, allowing the device to be secured in place, while rotation of the lid in the opposite direction causes air to be drawn up the air tube and deflates the balloon. The device further comprises an anti-drop cup that unfolds toward the distal end of the device as the device is removed from the stoma and is adapted to collect any leakage from the stoma during removal of the device.

### FIELD OF THE INVENTION

The present invention relates to the field of apparatuses for removal and collection of fluid or solid material from a stoma, especially temporary devices.

### BACKGROUND

According to the National Alliance of Wound Care and Ostomy, approximately 12,000 ostomy surgeries are performed each year in the United States. A stoma surgery may be required in patients with any of the following diseases or conditions: bowel cancer, gastrointestinal disorders such as Inflammatory Bowel Disease (IBD) or Crohn's disease, and trauma. A stoma surgery may be temporary or permanent. Colostomy, ileostomy and urostomy are three types of surgeries that create a stoma for excretion of fluid or feces from the body.

Several options are available for the removal of excrement from a stoma. Irrigation is a method of using an irrigation tube to flush warm water through the opening, causing the excrement to fall out. However, the long term use of this method has been discouraged by health professionals due to risk of perforation, mucosal burning, stricture, and loss of bowel tone. Another common option for excrement removal is an external ostomy pouch or bag connected to a catheter that is inserted into the stoma. However, such pouches generally require frequent changing, leaving the ostomate in a dilemma as to how to accomplish this change without excrement leaking onto his skin, body or surrounding area. It is advised by health professionals to clean the skin surrounding the stoma well in between bag changes so as to prevent infection and skin irritation, and in some cases it is preferable to apply ointments or creams.

However, many ostomates are unable to properly perform this cleaning and care, because they attempt to perform a bag change as fast as possible to avoid leakage from the stoma. In an effort to prevent leakage from the stoma, toilet paper is often placed over the stoma during bag changing, but such tissue does not stay in place and does not have the capacity to absorb all of the fluid or feces that may be excreted from the stoma. Therefore, a simple, inexpensive temporary device is needed that seals the stoma by collecting any excrement coming from the stoma. Such a device would need to stay firmly in place while allowing the ostomate time to perform necessary functions such as care for the skin around the area of the stoma, showering, and preparing to insert a new bag, without the concern of leakage. Such a device would allow the ostomate to choose when to insert and when to remove the device from his stoma according to his convenience and health needs, especially those related to the care of his stoma.

Devices exist for inserting inside a stoma that have internal balloons that may be inflated via a pump, piston, or air syringe such that the catheter is held firmly in place. However, such pumps may be large, difficult to use, and lack the ability for the user to control the amount of air entering the balloon. Therefore, a user may accidentally over-inflate a balloon, causing pain or damage to the area of his stoma. Likewise, such a balloon may be under inflated, leading the device to be insecure inside the stoma and thus causing unnecessary leakage. Such existing pumps may also cause pressure against the body and to the area of the stoma. The stoma is a non-sensitive part of the body due to its absence of nerves, so strong pressure applied to this area may cause damage that the ostomate is unaware of. Therefore, such pressure to this area should be carefully avoided.

For example, an air syringe is shown in U.S. Pat. No. 2,324,520 to Otis Lamson for an “Apparatus and method for closing abnormal openings in wall like members of the anatomy” that is used to inflate an internal inflatable blocking member inside the stoma. Such a syringe is difficult to use, as there is no mechanism for controlling the amount of air entering the air syringe. The device described in U.S. Pat. No. 5,569,216 to Kim for “Multipurpose colostomy device having balloons on an end thereof” is a multipurpose colostomy device that includes internal and external balloons, designed to fit in the stoma or rectum of a human. The balloons are inflated by the user repeatedly pushing first and second pistons. In addition to such repeated movement being cumbersome for the user, such pistons do not allow for the user to control of the amount of air that is entering the balloons.

There therefore exists a need for a simple, inexpensive temporary stoma evacuation device that prevents leakage from the stoma, stays in place while preventing damage to the ostomate, and is simple to operate, while overcoming at least some of the disadvantages of prior art systems and methods.

The disclosures of each of the publications mentioned in this section and in other sections of the specification, are hereby incorporated by reference, each in its entirety.

### SUMMARY

The present application describes new exemplary devices that may be inserted into a stoma and secured inside the stoma for temporary collection and ultimate removal of fluid or solids from the stoma, all of which is performed in a manner that prevents leakage. Such an exemplary device may comprise a cylindrical head portion incorporating a container at the proximal end of the device, a catheter fluidly connected to the container distally, and an inflatable balloon at the distal end of the catheter externally thereto, such that when the balloon is inflated, the catheter is secured inside the stoma.

In order to inflate the balloon in a simple and controlled manner, a novel pump is incorporated into the outer wall of the device. The device has a double walled structure, incorporating an annular volume between its inner and outer walls. The volume of air in the annular volume is fixed. The container can be covered by a lid attached to a longitudinal movable flat stick or piston element, such as a rod or a flat paddle-shaped element, that fits within and can move radially around the annular volume. The longitudinal cross sectional area of the piston element, namely the radial width and axial height, should substantially match the longitudinal cross section of the annular volume, such that rotation of the piston element around the annular volume sweeps air around the annular volume in front of the direction of motion of the piston element. A fixed barrier is located longitudinally down the length of the annular volume at one position in the circumference of the double wall, fitting between and separating the inner and outer walls. Such a fixed barrier or divider blocks essentially any air from flowing through the annular volume from one side of the barrier to the opposite side of the barrier. An orifice provided in the inner wall of the container at a position near the barrier, fluidly connects the annular volume to a passageway leading to the distal end region of the catheter, where the passageway is connected to the internal volume of the inflatable balloon.

The cylindrical head portion has a rotatable lid, for manual rotation by the user. The movable paddle is attached to the internal surface of the rotatable lid, such that rotation of the lid causes rotation of the attached paddle. The lid may align to the container in such a way that, when the lid covers the container, the paddle begins to move from an initial position on the side of the barrier opposite to the side where the orifice is located. Rotation of the lid in this manner causes air in front of the moving piston or paddle to be swept around the annular volume and out of the remote orifice down the passageway, thereby inflating the balloon.

The balloon is adapted for inflation inside the stoma and for deflation prior to removal of the device from the stoma. Inflation of the balloon causes the walls of the balloon to make contact with the inside walls of the stoma, and is thus used to secure the device inside the stoma. The volume of the annular space between the inner and outer parts of the double wall is predetermined such that the air in that volume is just sufficient to inflate the balloon to the desired extent, without danger of over-inflation.

Such devices may be useful as a temporary plug during bag changing, caring for the skin around the stoma, showering, or any other temporary situations in which an ostomate needs to contain the excrement and fluids being excreted from his stoma, without wearing the conventional attached collection bag. Such devices have simple construction, are easy to use, and are inexpensive to produce, allowing for disposability.

There is thus provided in accordance with an exemplary implementation of the devices described in this disclosure, a device for fixing a stoma, comprising:

(a) an annular volume in a head portion of the device, the annular volume formed between inner and outer walls,

(b) an airtight longitudinal barrier fixed within the annular volume,

(c) a fluid container positioned internal to the inner wall of the annular volume,

(d) a tube fluidly connected to the distal end of the container,

(e) an inflatable balloon disposed peripherally around the tube at its distal end region,

(f) a first orifice provided in a wall of the annular volume, proximal to the fixed barrier, the first orifice fluidly connecting the annular volume through a passageway to a second orifice opening into the inflatable balloon, and

(g) a rotatable lid fitting into the proximal opening of the container, to which is attached a paddle adapted to fit longitudinally within the annular volume, wherein rotation of the rotatable lid is configured to cause the paddle to sweep air in the annular volume out of the first orifice, through the passageway, and out of the second orifice, thereby inflating the balloon.

Variations of this device may further comprise, on the periphery of the container, a foldable cup of a flexible material adapted to collect leakage from the stoma during removal of the device from the stoma.

The device may operate such that rotation of the rotatable lid in either direction is less than one complete rotation of the lid. The mechanical forces created by the rotation of the rotatable lid in any degree of rotation should not applied to the stoma.

The device may comprise a uni-directional flap at the distal end of the tube, the flap adapted to prevent flow of material out of the tube. The device may have a pawl, a locking pin, or a ratchet adapted for securing the rotatable lid in a fixed position.

The passageway for air transfer may be either annular or tube-shaped. The annular volume of the device may be peripheral to the passageway, or the passageway may be peripheral to the annular volume. The annular volume may comprise a predetermined volume sufficient to inflate the balloon to fix the device in a stoma.

### BRIEF DESCRIPTION OF THE DRAWINGS

The present invention will be understood and appreciated more fully from the following detailed description, taken in conjunction with the drawings in which:

FIG. 1 shows a longitudinal cross section of an exemplary device while inserted into the stoma;

FIG. 2 shows an exploded view of a cylindrical head portion of an exemplary stoma evacuation device comprising a manual rotatable pump and air chamber;

FIG. 3 shows a longitudinal cross section of an exemplary device during removal from the stoma;

FIG. 4 shows a cross sectional top view of a cylindrical head portion of an exemplary device;

FIGS. 5A-5C show an exemplary method of inserting the device into a stoma;

FIGS. 6A-6C show an exemplary method of securing the device inside a stoma;

FIGS. 7A-7D show an exemplary method of removing the device from a stoma;

FIG. 8 shows a cross-section of an alternative implementation of the device with the cylindrical head portion having a tubular passageway; and

FIG. 9 shows a cross-section of an alternative implementation of the device with an annular passageway being positioned peripheral to the annular volume.

### DETAILED DESCRIPTION

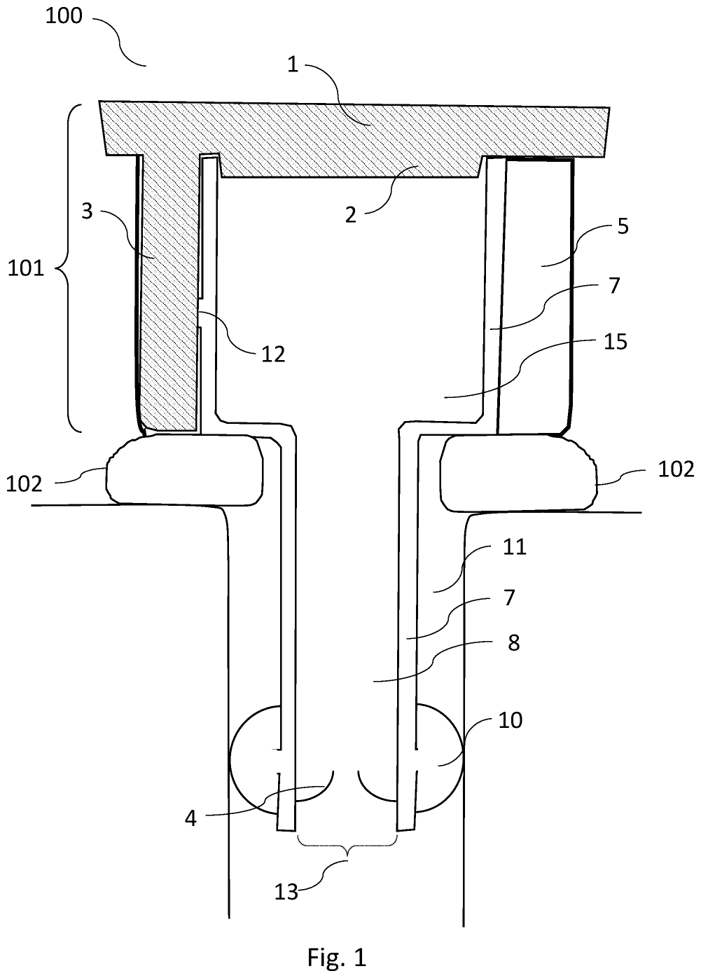

FIG. 1 shows a schematic longitudinal cross sectional drawing of an exemplary device 100 for insertion into a stoma, to enable temporary collection of fluids or solids from the stoma. FIG. 1 shows the device fully inserted and secured inside the stoma 102. The device 100 comprises a cylindrical head portion 101 comprising an annual volume 5 with a paddle element 3, constituting a user-operated rotary pump, and a container 15, a tube or catheter 8 for collection of material from the stoma, and an inflatable balloon 10 at the distal end of the cannula for securing the device in place. Excreted solid or fluid matter from the inner lumen of the stoma 11, i.e., from the intestinal segment in which the stoma has been created, enters through the catheter 8 at its open distal end 13, with the catheter being fluidly connected to the container 15 where the majority of the excrement and fluids are collected. The balloon 10 is shown fully inflated such that pressure exuded by the inflated balloon 10 is just sufficient to press against the inner walls of the stoma 11 and secure the device in place without causing pain or damage. The balloon 10 also provides a seal against leakage from the stoma.

The catheter 8 and container 15 together comprise a collection volume, for example, of the order of 200 cc for an adult sized device, that is sufficient to allow the ostomate enough time to perform personal functions before the device becomes full and thus requires removal from the stoma. The cylindrical head portion 101 is shown flush with the outer surface of the stoma 102, which is the fully inserted position of the device. If the skin surrounding the stoma is irritated, the device need not be inserted to be flush with the stoma 102 during collection. The device 100 may be manufactured in any size suitable for securing inside a stoma; one exemplary device has a catheter or tube length of 4 cm and a cylindrical head portion length of 2 cm and is suitable for use with a loop stoma. It is understood that the volume of the collection container and size and length of both container and tubing may be adapted for the individual situation. It is possible to manufacture a range of sizes and lengths to suite a variety of clinical scenarios and patients. The device may further comprise a one-way flow flap 4 inside the distal end of the catheter, allowing excrement/fluid to flow from the stoma into the catheter while the device is inside the stoma, but generally preventing excrement/fluid from flowing or leaking out of the open distal end of the catheter during removal of the device from the stoma.

The cylindrical head portion 101, located at the proximal end of the device, is shown comprising a manual rotation-driven pump having a rotatable lid 1 with an internal stopper 2, the rotatable lid 1 being attached to a paddle 3 that moves with rotation of the lid, and fits within the annular volume 5. An opening 12 is located between an annular volume 5 and a passageway 7 that allows air to flow from the annular volume 5 into the passageway 7 and vice versa, with an opening in the distal end region of the passageway opening into the balloon interior such that the air flow is used for inflation and deflation of the balloon 10. The moving paddle 3 is used to sweep air around the annular volume 5 and through the orifice 12, down the passageway 7 and into a second orifice for inflation of the balloon. The shoulder portion of the rotatable lid 2 may advantageously have a raised section that is matched to the internal shape of the top of the container 15 and fits therein, ensuring proper rotational movement of the moving radial wall 3 around the annular volume 5 while eliminating or minimizing escape of air between the lid 1, 2 and the top of the container. The orifice or opening 12 fluidly connects the annular volume 5 to the balloon 10 through the passageway 7, and allows for bidirectional flow of gas therein. Gas flowing through the passageway towards the distal end of the device causes inflation of the balloon, while deflation of the balloon causes gas to flow through the passageway towards the proximal end of the device.

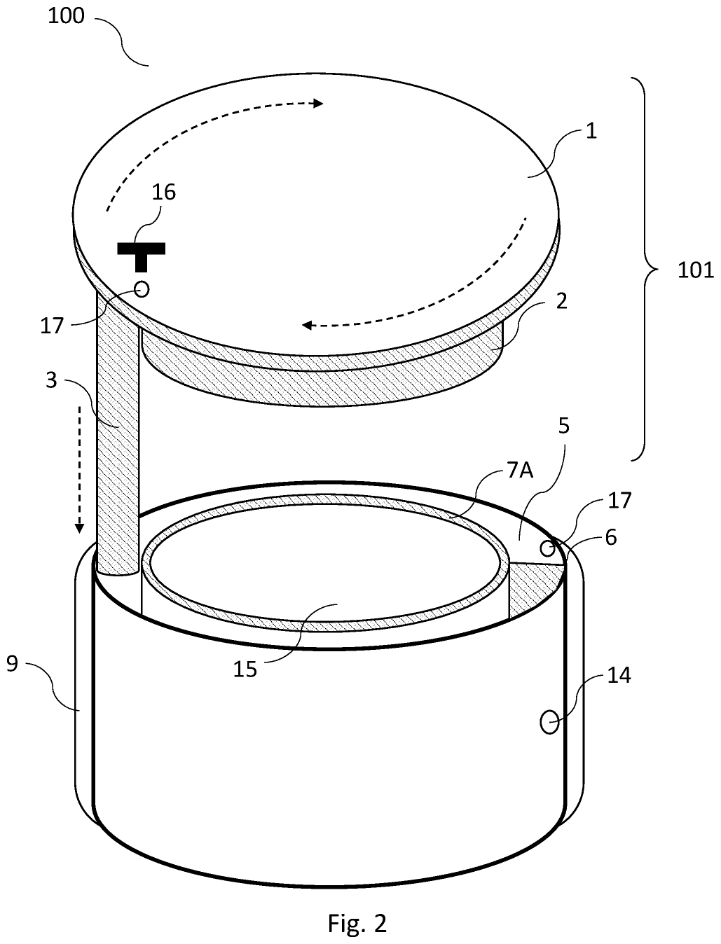

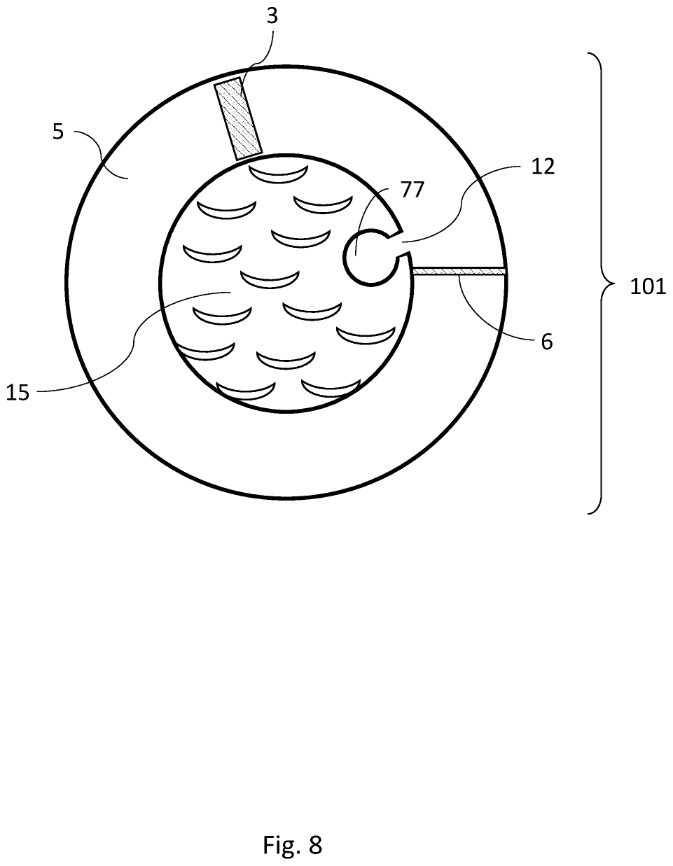

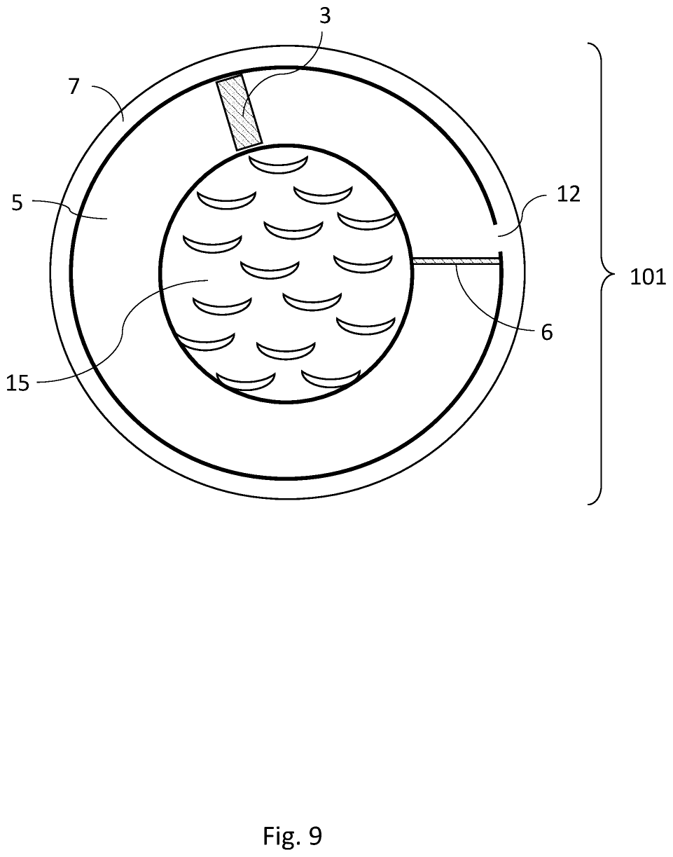

The opening 12 is of a sufficient size to provide enough air flow therethrough for efficient inflation and deflation of the balloon. The opening 12 may be located on the inner surface of the outer wall housing the annular volume 5, as shown in FIG. 1, with the passageway 7 radially inwards of the inner wall of the annular volume 5. Alternatively, the opening 12 and passageway 7 may be located on the outer part of the outer wall housing the annular volume 5, such that the upper section of the passageway 7 is peripheral to the annular volume 5, this configuration being shown in FIG. 9 hereinbelow. The passageway 7 may be annular as shown in FIG. 1. Alternatively, the passageway may be in the form of a tube, as shown in FIG. 8 below, so long as it provides sufficient fluid conduction to the balloon 10. Such a tubular passageway may be located either radially internal to the annular volume or external to the container. The passageway 7 shown in the exemplary device in FIG. 1 extends from the cylindrical head portion to the balloon 10 at the distal end of the device. The air pump comprising the rotatable lid 1, 2 and paddle 3, and annular volume 5 is more fully described below in accordance with FIG. 2.

FIG. 2 shows an isometric exploded view of the cylindrical head portion of the device of FIG. 1, illustrating the manual rotation-driven pump comprising the annular volume 5 for inflation and deflation of the balloon. The annular volume 5, situated between inner and outer walls of the device, is calculated to contain a predetermined quantity of gas that is sufficient to inflate the balloon (not shown in FIG. 1) to the desired extent. The rotatable lid 1 may be rotated in both directions by the user. The rotatable lid 1 fits into the proximal end of the inner container 15 via its bottom shoulder portion 2 such that the container is sealed and the shoulder portion 2 does not allow any excrement/fluid to flow out of the proximal end of the container 15. However, an alternative embodiment may provide a removable stopper in the rotatable lid 1 (not shown in Figures), providing the user with an option of attaching an external bag to the proximal end of the device. This embodiment would increase the collection of excrement to beyond the volume of the container 15 in cases of heavy flow. The rotatable lid 1 is attached to the paddle 3 such that a full rotation of the rotatable lid 1 causes the paddle 3 to rotate through the annular circumference of the annular volume 5 from one side of the fixed barrier 6 to the other side of the fixed barrier 6. Rotation of the rotatable lid 1 in one direction causes the paddle 3 to move around the annular volume until it meets one side of the fixed barrier 6, which stops its rotation. This causes air to flow through the orifice in the inner wall (not visible in FIG. 2) down the passageway, the closed top of which, 7A, is shown radially inward of the annular volume 5 in this figure, and to inflate the balloon, allowing the device to be secured in place inside the stoma, while rotation of the lid 1 in the opposite direction causes the paddle 3 to move to the opposite side of the fixed barrier 6; this causes air to be drawn up via the passageway into the annular volume 5 and deflates the balloon, allowing for removal or insertion of the device out of or into the stoma. Optionally, a small outer orifice 14 containing a one-way flap or valve may be located in the outer wall of the cylindrical head portion, allowing air to flow from the annular volume to the outside environment and keeping air pressure within the annular volume 5 at a desired level.

The capacity of the annular volume 5 is calculated to inflate the balloon 10 to the size required for securing inside the stoma. The paddle 3 essentially fills the longitudinal width and height of the annular volume 5, in effect sealing the space between the paddle 3 and the limiting surfaces of the annular volume 5, preventing air in the air chamber 5 from leaking past the moving paddle 3. Such a moving paddle 3 may be a rod or piston of cylindrical rod shape, cubic shape, rectangular shape, or any other shape that matches the cross-section of the annular volume 5 and yet allows for movement of the paddle 3 and the sweeping of air through the annular space 5. The fixed barrier 6 blocks air from flowing through the air chamber 5 from one side of the fixed barrier 6 to the opposite side of the fixed barrier 6.

After a rotation of the lid 1 is complete, such that the paddle 3reaches the fixed barrier 6, a locking pin 16 may be inserted into a pin orifice 17 to secure the lid to this fixed position. This securing mechanism prevents air pressure from the balloon 10 from leaking back out through the passageway 7 and rotating the paddle 3 in the direction that causes deflation. Although a locking pin 16 is shown in FIG. 2, it is to be understood that any locking mechanism such as a ratchet, a snap, a locking pin, or a pawl may be used to secure the lid to this fixed position. Alternatively, the friction between the paddle 3 and the annular space 5 may be sufficient to keep the paddle 3 in its position against the fixed barrier 6. The materials and dimensions may be determined to provide enough friction to overcome air pressure forces leaking upwards from the balloon 10 and to keep the paddle 3 in position after rotation of the lid 1, but to still provide ease of rotation of the lid 1 for the user.

The exemplary device shown in FIG. 2 is configured such that a single rotation of the rotatable lid 1 starting with the moving paddle 3 at one side of the fixed barrier 6 and culminating in the paddle 3 reaching the other side of the fixed barrier 6, for example a 340 degree turn, provides the volume of air sufficient to inflate the balloon 10 to an adequate size for securing inside the stoma. Likewise, a rotation of the lid 1 in the opposite direction is sufficient to deflate the balloon 10 and allow removal of the device 100 from the stoma. In addition to providing ease of use in avoiding the repeated pumping action of some prior art systems, this eliminates the user's concern of over-inflation or under-inflation of the balloon 10, because the moving paddle 3 reaching the fixed barrier 6 signals to the user that the balloon is inflated to the correct predetermined fixed volume, which is determined by the capacity of the annular volume 5. Such a predetermined fixed volume is determined to be such that it will not cause damage to the stoma from over-inflation of the balloon, but will secure the device 100 firmly. The annular volume 5 and balloon are of sizes that correspond to this fixed volume. The ostomate should be able to feel when the paddle 3 makes contact with the fixed barrier 6 and thus he should know when the balloon is full and the device 100 is secured in place. Likewise, when the paddle 3 makes contact with the opposite side of the fixed barrier 6, the ostomate may be confident that the balloon has deflated to an amount sufficient for easy removal of the device 100 from the stoma. Since inflation of the balloon is limited by the force of turning the pump and by the volume of air inside the annular volume 5, over-inflation of the balloon 10 which could cause discomfort or damage to the stoma is prevented. Likewise, under-inflation of the balloon 10 would result in movement of the device 100 within the stoma, probably leading to leakage of excrement.

Furthermore, the exemplary device 100 is adapted such that rotation of the lid 1 eliminates unnecessary pressure from being applied to the stoma during inflation of the balloon, as may occur in some prior art devices that apply a pumping motion against the body of the ostomate during actuation of the pump. The force from the rotation of the lid 1 is not transferred as a mechanical force to the stoma but merely is used to inflate the balloon. The rotatable pump is designed in a manner such that essentially no pressure is applied to the skin around the stoma, which is an area sensitive to damage.

Also shown in FIG. 2 is a foldable cup 9 at the periphery of the cylindrical head portion 101 that may be unfolded toward the distal end of the device 100 during removal of the device 100 from the stoma for collection of leakage during removal. Such a foldable cup 9 may be made of a flexible, watertight material such as rubber, silicon, latex or other pliable material. Such a foldable cup 9 is adapted to collect leakage from the stoma during removal, thus preventing soiling of the skin surrounding the stoma, the body, or the area surrounding the ostomate. As described previously in accordance with FIG. 1, the device 100 may further comprise a one-way flow flap 4 inside the distal end of the catheter 8, allowing excrement to flow from the stoma 11 into the catheter 8 while the device 100 is inside the stoma 11, but preventing excrement from flowing or leaking out of the open distal end of the catheter 13 during removal of the device 100 from the stoma 11. As described previously, an alternative embodiment provides a stopper at the proximal end of the device (not shown in Figures) that may be removed and a bag that may be placed over the proximal end of the device 100, allowing the excrement to flow into the bag. As the user removes such a device 100 from his stoma 11, he may tilt the device 100 downwards such that gravity causes much of the excrement and fluid in the catheter 8 and in the container 15 to flow into the bag, reducing the chance of excrement leaking from the open end of the catheter 13. Thus, all three of these implementations allow for a cleaner removal process of the device from the stoma. When the device 100 is removed, usually when it is full or mostly full, the device 100 may be disposed of along with its collected contents.

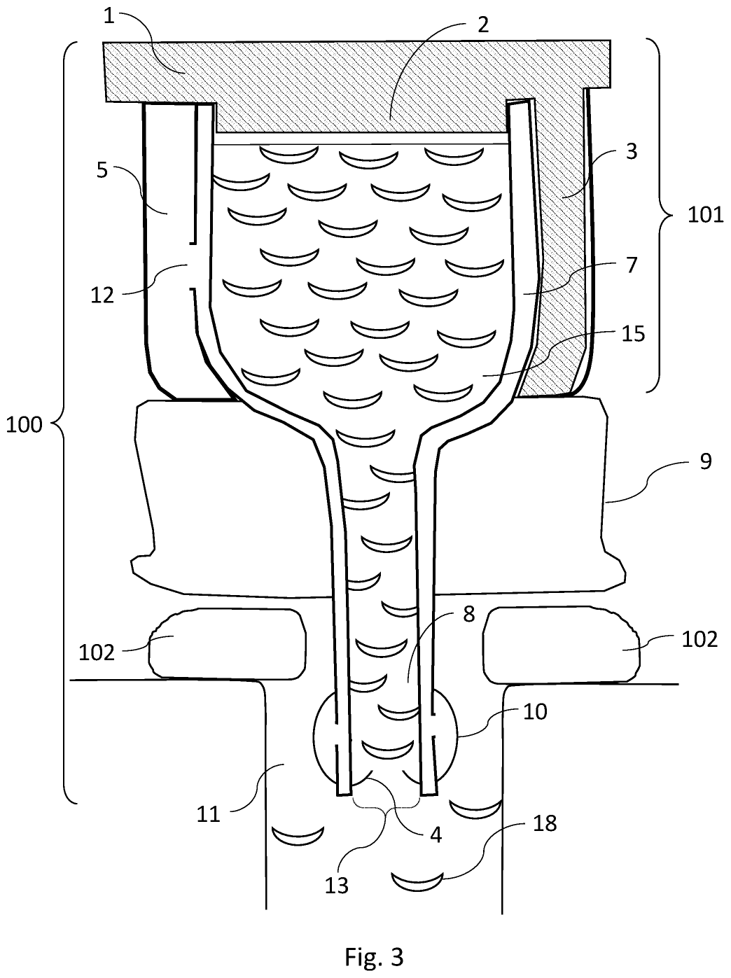

FIG. 3 shows a schematic drawing of the longitudinal cross section of the device during removal from the stoma. The paddle 3 is shown on the opposite side of the annular volume 5 from its location in Fig.1, making contact with the opposite side of the fixed barrier 6 compared with FIG. 1. The balloon 10 is deflated to allow for easy removal of the device 100. Air has been drawn from the balloon 10 up the passageway 7 and into the annular volume 5 through opening 12, deflating the balloon 10. The balloon 10 is deflated sufficiently to allow for easy removal of the device 100 from the stoma 11. The foldable cup 9 is fully unfolded toward the distal end of the device such that it is peripheral to the catheter 8 to collect any leakage from the lumen of the stoma 11 during removal. The catheter 8 and container 15 are full or nearly full of excrement 18.

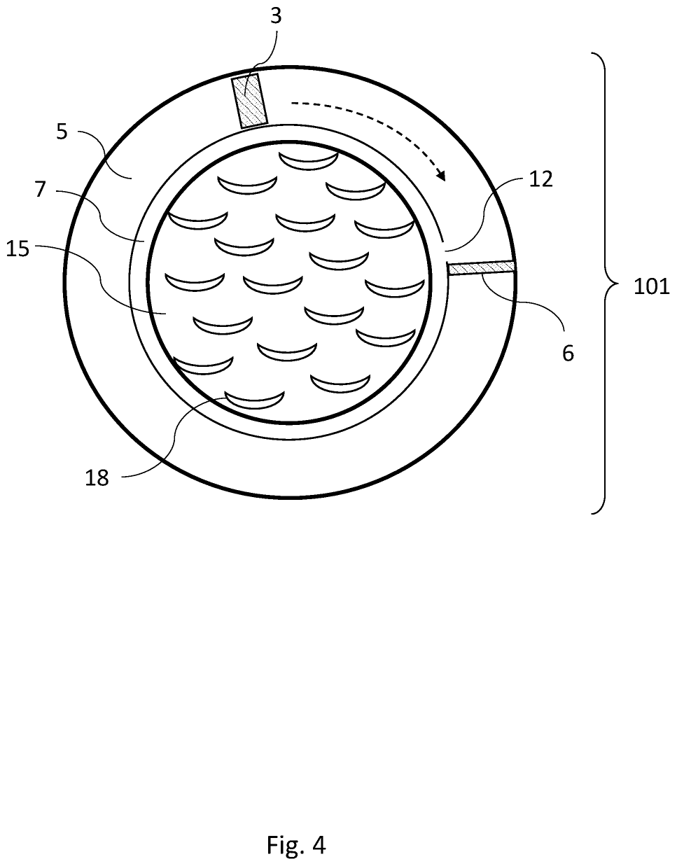

FIG. 4 shows a schematic cross-sectional top view of the cylindrical head portion 101 of the device of FIGS. 1 to 3. The container 15 is for collection of feces and fluid 18 drawn in through the catheter or tubing. The annular volume 5 includes the fixed barrier 6 and is positioned outside the passageway 7 located peripheral to the container 15. The opening 12 connects the annular volume 5 to the passageway 7. The movable paddle 3 fits within the annular volume 5.

The cylindrical head portion 101 is most conveniently implemented as a right circular cylinder, though it may also be tapered such that it does not maintain the same diameter at all locations along its longitudinal length. The container 15 may be of any suitable shape and size for collecting the needed volume of excrement. Furthermore, although a circular shape is most convenient in manufacture and use, the cylindrical head portion 101 may be formed with an oval or other shape, provided that a suitable mechanism is provided to allow for a non-circular pump motion.

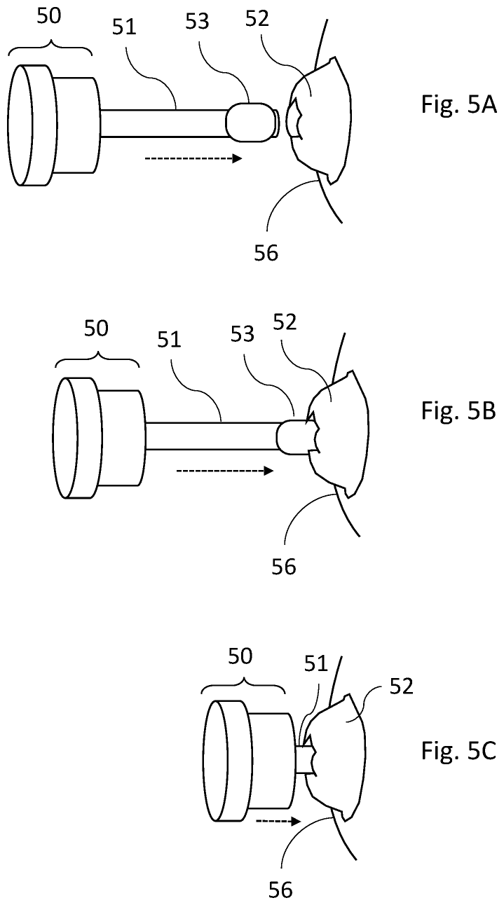

FIGS. 5A to 5C show a method of inserting the device 100 shown in FIGS. 1 to 4 into a stoma 52. The device includes the cylindrical head portion 50, the tubing or catheter 51, and the uninflated balloon 53 at the distal end of the tubing 51. After cleaning the skin area 56 around the stoma 52, the device is inserted inside the stoma 52, as shown in FIGS. 5A and 5B, until the cylindrical head portion 50 is essentially flush with the stoma 52 as shown in FIG. 5C. The balloon 53 is in its deflated state during insertion. Once inserted and fixed into the stoma 52 by inflation of the balloon 53, the device may begin collection of feces through the catheter 51 into the combined volume of the catheter 51 and container, the container being housed in the cylindrical head portion 50.

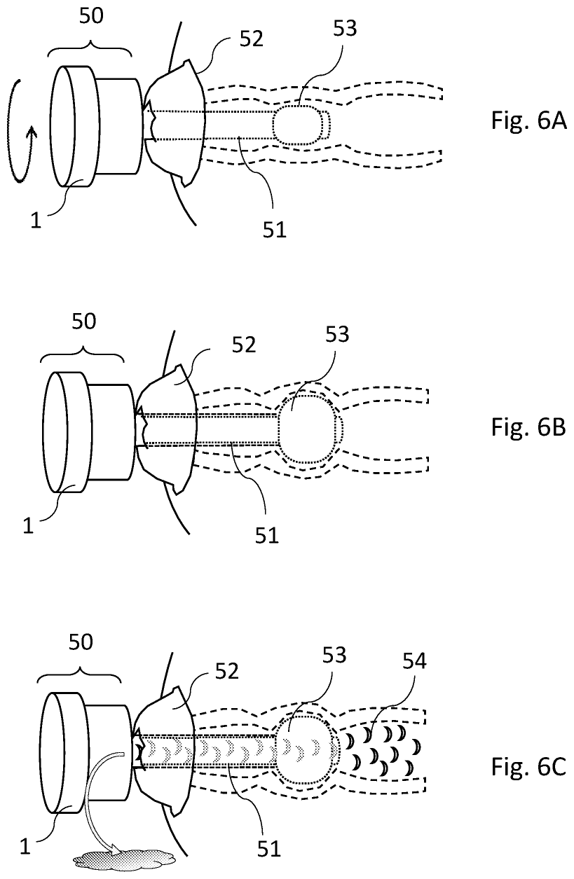

FIGS. 6A to 6C show a method of securing the device inside a stoma 52. Once the device is placed fully inside the stoma 52 such that the cylindrical head portion 50 of the device is flush against the stoma 52, as shown in FIG. 6A, the lid 1 is rotated by the user, causing the balloon 53 to inflate, as shown in FIG. 6B. The balloon 53 presses against the inner walls of the stoma 52, i.e., against the inner walls of the intestinal segment in which the stoma has been created, and secures the device in place, as shown in FIG. 6B. Once the device is anchored in place, it can collect excreted matter 54 through the catheter 51, as shown in FIG. 6C.

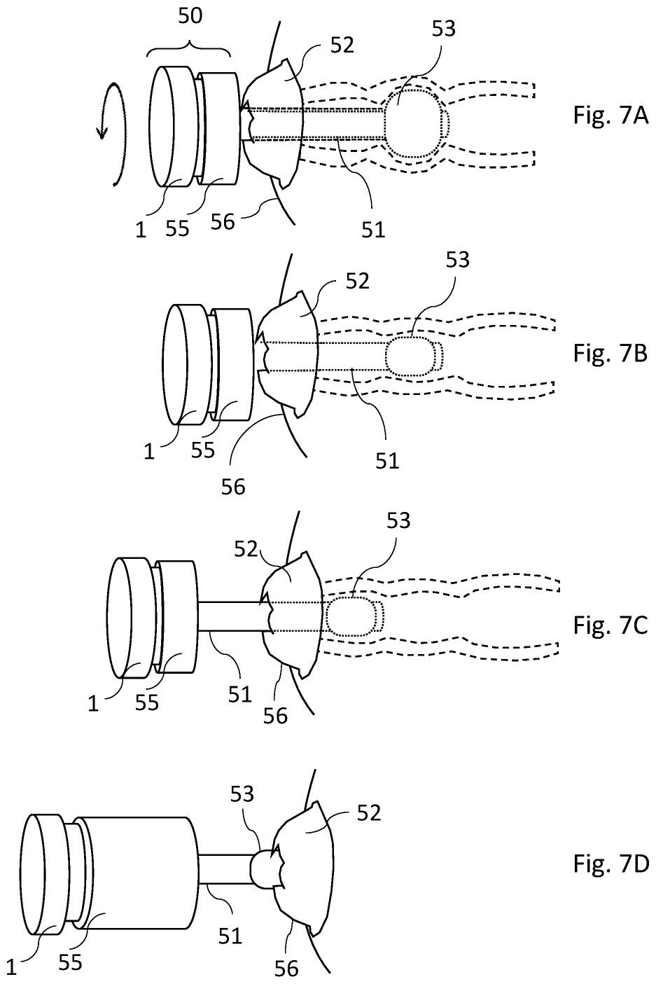

A method of removing the device from the stoma 52 is shown in FIGS. 7A to 7D. Before removal, the user may place a sticker on the skin surrounding the stoma that functions as an adhesive for securing a bag. The exemplary device of FIGS. 7A to 7D may be removed without interfering with such a sticker. To prepare for the removal of the device from the stoma 52, as shown in FIG. 7A, the user turns the rotatable pump lid 1 in the cylindrical head portion 50 in the opposite direction to that used for inflation, causing the balloon 53 to deflate, as shown in FIG. 7B. Alternatively, the user may release a securing mechanism such as a snap, latch, or locking pin, causing the lid 1 to rotate by itself in the direction that causes deflation of the balloon 53. Once the balloon 53 is deflated, the device may be safely withdrawn from the stoma 52 with its collected contents, as shown in FIG. 7C. In FIGS. 7A-7C the foldable cup 55 is shown as folded upwards so that it is peripheral to the cylindrical head portion, taking up only a small amount of space when not in use. During removal of the device from the stoma 52, as the cylindrical head portion 50 moves away from the skin level of the stoma 56, the user may roll down the foldable cup 55 towards the stoma 52 as shown in FIG. 7D, to collect additional droplets that might exist in the area of the stoma. After use, the device may be discarded along with its collected contents.

FIG. 8 shows a schematic cross section of the cylindrical head portion 101 of an alternative implementation of the device, in which a tubular passageway 77 is used for conveying the air from the orifice 12 in a wall of the rotary pump volume 5, to the balloon 10. This is an alternative to the annular passageway 7 of FIG. 4. The shape of the container portion 15 is changed accordingly to accommodate the passageway 77. The other elements of the device are shown in the same manner as in FIG. 4.

FIG. 9 shows a schematic cross-section of the cylindrical head portion 101 of a further alternative implementation of the device, in which the passageway 7 is positioned peripheral or external to the annular volume 5, such that the opening 12 is located between the passageway 7 and the annular volume 5. The fixed divider 6, the movable paddle 3, and the container operate as in FIG. 4. An alternative implementation of the passage could be in the form of a tubular passageway disposed outside of the outer wall of the annular volume.

Although the most conventional and simplest implementation of the device will be constructing the cylindrical head portion 101 with a circular circumference, it should be understood that the term cylindrical throughout this disclosure and as claimed may also include shapes other than a circular shape, such as those with an ellipsoidal or oval circumference, with appropriate mechanisms to enable the movable paddle to follow the shape of the annular volume. Furthermore, although the most common implementation of the device uses a cylindrical shape of uniform diameter along the longitudinal dimension, it is to be understood that tapered shapes in which the diameter is not constant throughout the longitudinal length of the shape may also be used.

It is appreciated by persons skilled in the art that the present invention is not limited by what has been particularly shown and described hereinabove. Rather the scope of the present invention includes both combinations and subcombinations of various features described hereinabove as well as variations and modifications thereto which would occur to a person of skill in the art upon reading the above description and which are not in the prior art.

1. (canceled)

2. A device according to claim 11, further comprising, on the periphery of said container, a foldable cup of a flexible material adapted to collect leakage from said stoma during removal of said device from said stoma.

3. A device according to claim 11, wherein said rotation of said piston in either direction is less than one complete rotation.

4. A device according to claim 11, wherein said passageway is either annular or tube-shaped.

5. (canceled)

6. A device according to claim 11, wherein the use of a rotating piston to inflate said balloon is such that mechanical forces created from the rotation of said piston are not applied to said stoma.

7. A device according to claim 11, further comprising a uni-directional flap at the distal end of said tube, said flap adapted to prevent flow of material out of said tube.

8. A device according to claim 12, further comprising at least one of (i) a pawl, (ii) a locking pin, and (iii) a ratchet adapted for securing said rotatable lid in a fixed position.

9. A device according to claim 11, wherein said annular shaped volumetric space is peripheral to said passageway.

10. A device according to claim 11, wherein said passageway is peripheral to said annular shaped volumetric space.

11. A device for collecting waste from a stoma of a subject, said device comprising:
a fluid container for collecting waste material from the stoma;
a tube fluidly connected to said fluid container, and adapted to transfer waste from the stoma;
an inflatable balloon disposed peripherally around said tube;
an annular shaped volumetric space adapted to hold air for inflation of said balloon;
an inflation passageway connecting an end region of said annular volumetric space to said balloon; and
a movable piston located in said volumetric space, such that rotation of said piston through said annular shaped volumetric space towards said first end region sweeps the air in said annular shaped volumetric space through said inflation passageway and into said balloon,
wherein said annular shaped volumetric space has a predetermined volume sufficient that a single rotary sweep of said piston inflates the balloon to a level that fixes said tube safely into the stoma.

12. A device according to claim 11, further comprising a rotatable lid, said piston being attached to said rotatable lid, such that rotation of said rotatable lid generates said rotation of said piston.

13. A device according to claim 11, further comprising an airtight barrier fixed within said annular shaped volumetric space, defining the end region of said annular shaped volumetric space, and adapted to limit the rotation of said piston to a single revolution.
